(19) United States
(12) Patent Application Publication
(10) Pub. No.: US 2024/0293149 A1
(43) Pub. Date: Sep. 5, 2024

(54) STEERABLE INTRODUCER DEVICES, SYSTEMS, AND METHODS

(71) Applicant: Merit Medical Systems, Inc., South Jordan, UT (US)

(72) Inventor: Jeremy Snow, South Jordan, UT (US)

(21) Appl. No.: 18/592,936

(22) Filed: Mar. 1, 2024

Related U.S. Application Data
(60) Provisional application No. 63/488,279, filed on Mar. 3, 2023.

(51) Int. Cl. A61B 17/34; A61B 17/00

(52) U.S. Cl. CPC A61B 17/3417; A61B 17/3421; A61B 2017/00367; A61B 2017/3447; A61B 2017/3454

(57) ABSTRACT

Embodiments of a steerable device, and methods for using said device, for introducing medical devices into a patient's body is disclosed. The device may be inserted into a patient's body in a linear configuration, transitioned into an articulated configuration and then used to traverse to a precise location. The device may be articulated into a variety of configurations via an actuation portion of the handle.

### RELATED APPLICATIONS

This application claims priority to U.S. Provisional Application No. 63/488,279, filed on Mar. 3, 2023 and titled, “Steerable Introducer Devices, Systems, and Methods,” which is hereby incorporated by reference in its entirety.

### TECHNICAL FIELD

The present disclosure relates generally to devices and methods for inserting a medical introducer in a patient, for example in preparation for taking a biopsy. More particularly, some embodiments relate to navigation and steerability of a medical device introducer through a patient's body.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is a perspective view of an exemplary embodiment of a steerable introducer device.

FIG. 2 is a perspective view of an exemplary embodiment of a translating portion and a first conduit of the steerable introducer device of FIG. 1.

FIG. 3 is a side view of an exemplary embodiment of a distal end of the first conduit of the steerable introducer device of FIG. 1.

FIG. 4 is a perspective view of an exemplary embodiment of a handle, including an adapter portion, and a second conduit of the steerable introducer device of FIG. 1.

FIG. 5 is a side view of an exemplary embodiment of a distal end of the second conduit of the steerable introducer device of FIG. 1.

FIG. 6 is a perspective view of an exemplary embodiment of the first conduit, the second conduit, and the handle, including the adapter portion and the translating portion, of the steerable introducer device of FIG. 1.

FIG. 7 is a cutaway view of an exemplary embodiment of the first conduit, the second conduit, and the handle, including the adapter portion, the translating portion, and the actuating portion, of the steerable introducer device of FIG. 1.

FIG. 8 is a perspective view of an exemplary embodiment of the second conduit and the handle, including the adapter portion and the actuating portion, of the steerable introducer device of FIG. 1.

FIG. 9 is a perspective view of an exemplary embodiment of the distal end of the steerable introducer device of FIG. 1.

FIG. 10 is a perspective view of an exemplary embodiment of the steerable introducer device of FIG. 1.

FIG. 11 is a cutaway view of an exemplary embodiment of the steerable introducer device of FIG. 1 in a linear configuration.

FIG. 12 is a cutaway view of an exemplary embodiment of the steerable introducer device of FIG. 1 in a first articulated configuration.

FIG. 13 is a cutaway view of an exemplary embodiment of the steerable introducer device of FIG. 1 in a second articulated configuration.

### DETAILED DESCRIPTION

In certain instances, a soft tissue lesion (e.g., tumor) may grow within a patient at various locations, such as breast, liver, lung, kidney, spleen, prostate, lymph nodes, etc. Sampling of a small portion of the lesion to determine the type of lesion such that a treatment regimen can be planned and implemented may be recommended to the patient. In some embodiments, the tissue sampling can be performed utilizing a biopsy device, or a biopsy introducer device, that is inserted into the lesion.

To accomplish this, it can be necessary for a practitioner to accurately locate, and precisely access, a lesion that may reside within a complex anatomy. Typically, before and/or during the biopsy procedure, a form of radiography is used to locate the lesion, as well as to obtain a mapping of nearby and relevant anatomy. Following, or concurrently with, identification and mapping of the lesion, a practitioner will typically introduce a type of incisional biopsy device, or biopsy devices, that can be used to sever and retrieve a tissue sample.

Given the above-mentioned variability in bodily locations in which a lesion can occur, together with the high individualization and variance in morphology at the lesion and surrounding anatomy, maneuverability and precision may facilitate use of a biopsy device, or a biopsy device introducer. As compared to a rigid, linear cannula or stylet, percutaneously inserted to the lesion, devices within the scope of this disclosure may be configured to provide maneuverability and precision in directing such a device.

The disclosure and embodiments in this application include methods, mechanisms, and components for adding steerability, maneuverability, and precision to such a biopsy device, or biopsy introducer device, as described throughout the application.

The components of the embodiments as generally described and illustrated in the figures herein can be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

The phrase “coupled to” is broad enough to refer to any suitable coupling or other form of interaction between two or more entities, including mechanical, fluidic and thermal interaction. Thus, two components may be coupled to each other even though they are not in direct contact with each other. The phrases “attached to” or “attached directly to” refer to interaction between two or more entities which are in direct contact with each other and/or are separated from each other only by a fastener of any suitable variety (e.g., mounting hardware or an adhesive). The phrase “fluid communication” is used in its ordinary sense, and is broad enough to refer to arrangements in which a fluid (e.g., a gas or a liquid) can flow from one element to another element when the elements are in fluid communication with each other.

The terms “proximal” and “distal” are opposite directional terms. For example, the distal end of a device or component is the end of the component that is furthest from the practitioner during ordinary use. The proximal end refers to the opposite end, or the end nearest the practitioner during ordinary use.

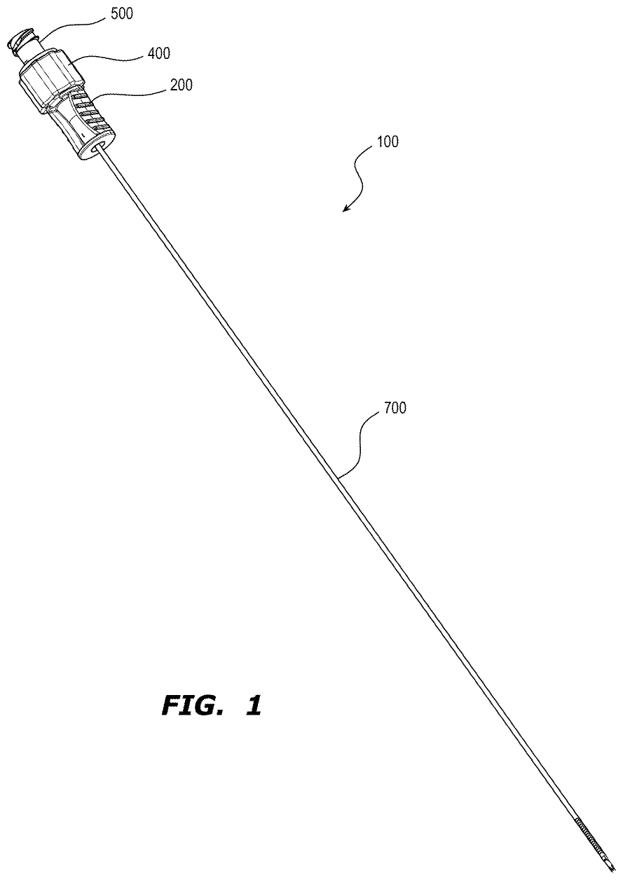

Turning to the drawings, FIG. 1 shows an exemplary embodiment of a steerable introducer device 100, or a steerable medical device for introducing medical instruments. Generally, the steerable introducer device 100 may include a handle 200, a first conduit 600, and a second conduit 700. The handle 200 may comprise a body portion 202, a slot portion 240, a bore portion 260, a translating portion 300, an actuating portion 400, and an adapter portion 500. The body portion 202 may comprise a grip portion 204 and grip ridges 206. The slot portion 240 may comprise slot protrusions 242 and a slot space 244. The bore portion 260 may comprise a proximal funnel 262, a main bore 264, and a distal funnel 266. The translating portion 300 may comprise a body portion 302 and a bore portion 320. The body portion 302 may comprise threads 304. The bore portion 320 may comprise a funnel portion 322 and a main bore portion 324. The actuating portion 400 may comprise an exterior 402 and threads 420. The exterior 402 may comprise ridges 404. The adapter portion 500 may comprise a body portion 502, a slot portion 520 and a bore portion 540. The body portion 502 may comprise threads 504. The slot portion 520 may comprise slot protrusions 522 and a slot space 524. First conduit 600 may comprise a proximal end 602, a main body 620, slots 640, and a distal end 660. Main body 620 may comprise a bore 622. Slots 640 may comprise an enlarged portion 642 and a function portion 644. Distal end 660 may comprise a protrusion 662. Second conduit 700 may comprise a proximal end 702, a main body 720, slots 740, a cut portion 760 and a distal end 780. Main body 720 may comprise a bore 722. Slots 740 may comprise an enlarged portion 742 and a function portion 744. Distal end 780 may comprise a cutting tip 782.

In various embodiments, steerable introducer devices 100 utilizing any combination of the components outlined above may be part of an assembly or device system that may or may not further include a second, elongate medical device.

In alternative embodiments, the steerable introducer device 100 and/or device system may or may not comprise a protective cover for a sharp, cutting edge of the insertion device.

In alternative embodiments, the system comprising the components may be included in various types of packaging or transportation and organizational components.

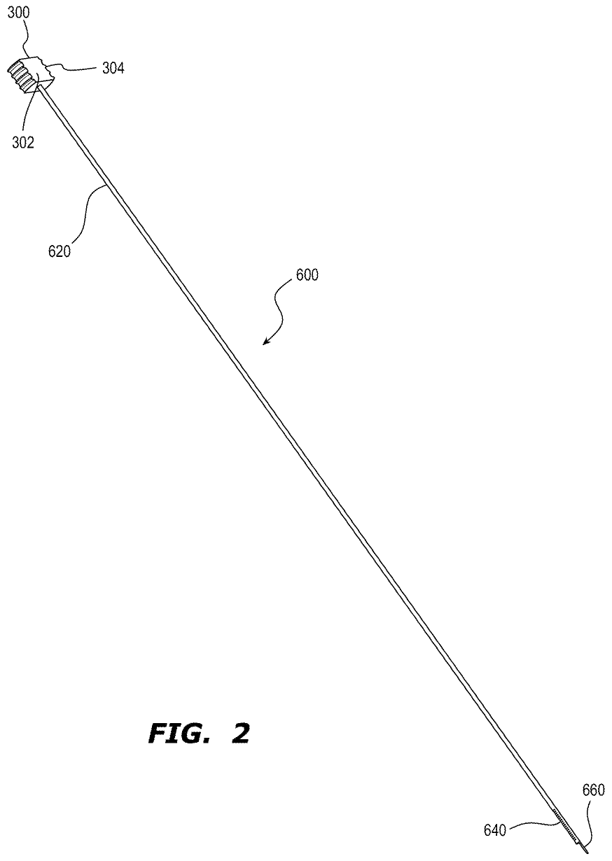

In an exemplary embodiment, FIGS. 2, 3, and 7 illustrate translating portion 302 and first conduit 600. Translating portion 300 may comprise body portion 302 and a bore portion 320. Body portion 302 may be a variety of shapes, including a rectangular shape, with rounded, lateral sides comprising threads 304. Threads 304 may be of any pitch and may be configured or tuned to displace the translating portion 302 a desired amount.

Translating portion 300 may further comprise bore portion 320 comprising funnel portion 322 and main bore portion 324. Main bore portion 324 may serve to couple the proximal end 602 of first conduit 600 such that translating portion 300 and first conduit 600 may be displaced together while maintaining a fixed, continuous lumen through both components.

First conduit 600 may further comprise a main body 620 with a continuous bore 622 extending from proximal end 602 to distal end 660. Distal end 660 may comprise a protrusion 662 that extends distally from the main body 620. First conduit 600 may further comprise slots 640 in the side wall adjacent to the distal end 660.

First conduit 600 may be formed from any suitable, biocompatible, rigid material.

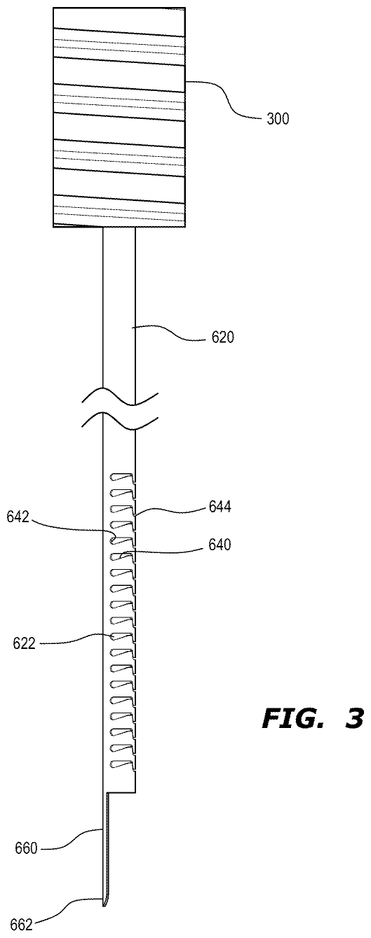

FIG. 3 illustrates an exemplary embodiment of the distal end 660 and slots 640 of first conduit 600. Slots 640 can be formed in the side wall of first conduit 600, and extend radially completely through the side wall of main body 620. Slots 640 may further comprise enlarged portion 642, and function portion 644. Function portion 644 may be non-linear with respect to enlarged portion 642, and may comprise a curved portion, or bend, in the slot as the slot traces around the surface of main body 620.

Distal end 660 may further comprise protrusion 662 that extends distally from a distal end of the main body portion 620. Protrusion 662 may be an extension of the main body where the cross-sectional area of the protrusion 662 is less than the cross-sectional area of the main body 620. Protrusion 662 may extend from a portion of the main body 620 that is radially opposite the function portion 644 of slots 640.

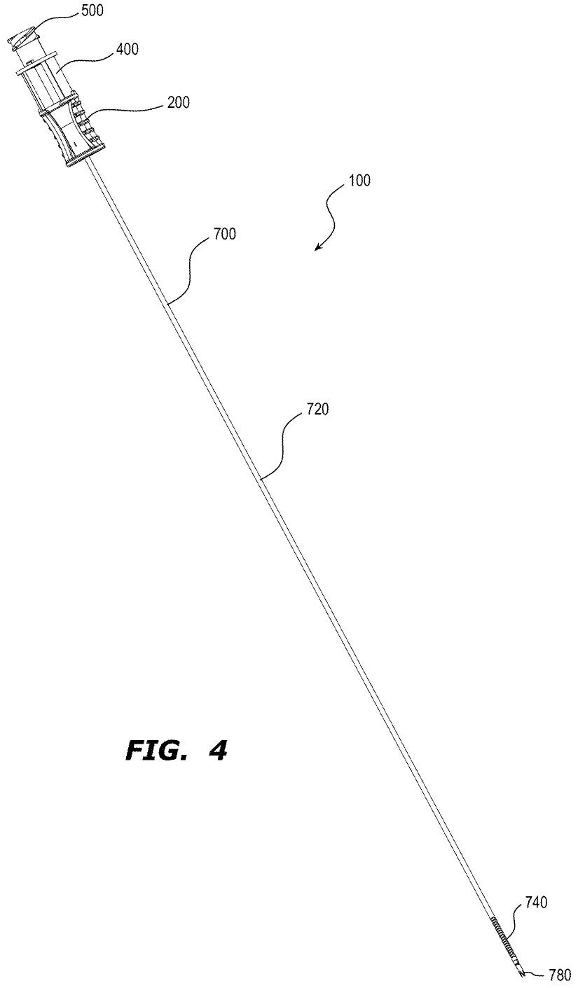

FIGS. 4, 5, 6, and 7 illustrate an exemplary embodiment of the second conduit 700 and handle 200, including body portion 202, slot portion 240, bore portion 260, and adapter portion 500. Body portion 202 may comprise a grip portion 204 configured to be held by a user of the device. Grip ridges 206 may be disposed on the exterior of the handle, and provide additional support, grip surface area, and/or ease-of-use for the user. Bore portion 260 may further comprise a proximal funnel 262, a main bore 264, and a distal funnel 266.

Second conduit 700 may comprise a proximal end 702, a main body 720, slots 740, cut portion 760, and distal end 780. Proximal end 702 of second conduit 700 may be fixedly attached to the handle 200. Proximal end 702 of second conduit 700 may be fixedly coupled to the main bore 264 of the bore portion 260 of the handle 200.

Second conduit 700 may be formed from any suitable, biocompatible, rigid material.

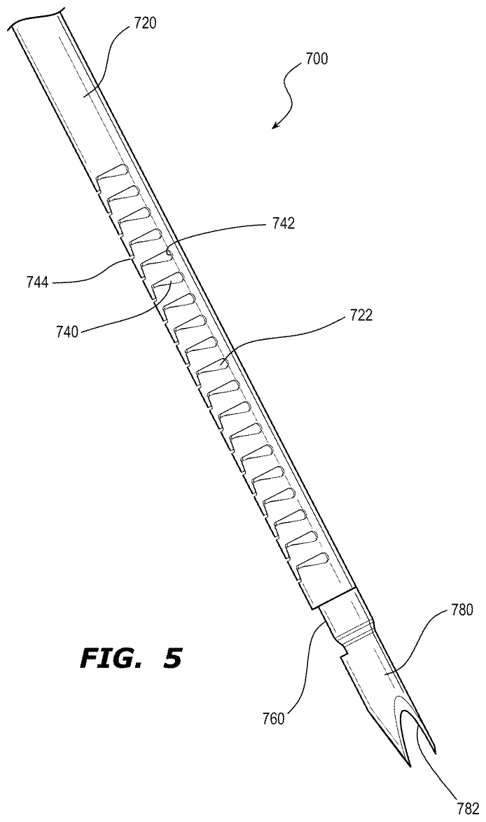

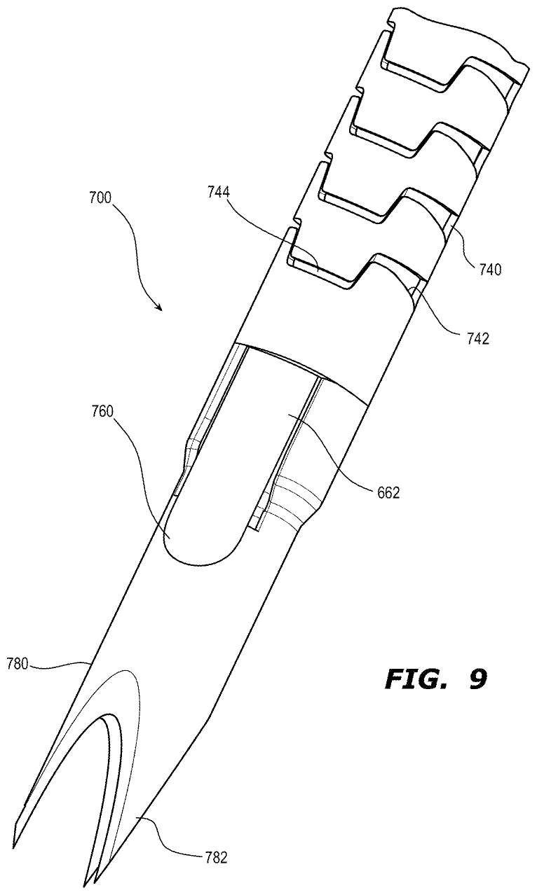

FIGS. 5 and 9 illustrate an exemplary embodiment of the main body 720, slots 740, cut portion 760, and distal end 780 of the second conduit 700. Slots 740 can be formed in the side wall of second conduit 700 and extend radially entirely through the side wall of main body 720. Slots 740 may further comprise enlarged portion 742, and function portion 744. Function portion 744 may be non-linear with respect to enlarged portion 742, and may comprise a curved portion, or bend, in the slot as the slot traces around the surface of main body 720.

Cut portion 760 may comprise a cutaway portion extending through the sidewall. In some embodiments, cut portion 760 may be configured to receive protrusion 662 in distal end 660 of first conduit 600. In some embodiments, second conduit 700 and first conduit 600 may be fixedly coupled together at cut portion 760.

Distal end 780 of second conduit 700 may further comprise a cutting tip 782 that may include a trocar, or multiple, cutting prongs a the most distal end of the device. One of ordinary skill in the art, having the benefit of this disclosure, will be able to design and implement various suitable cuttings tips for the device.

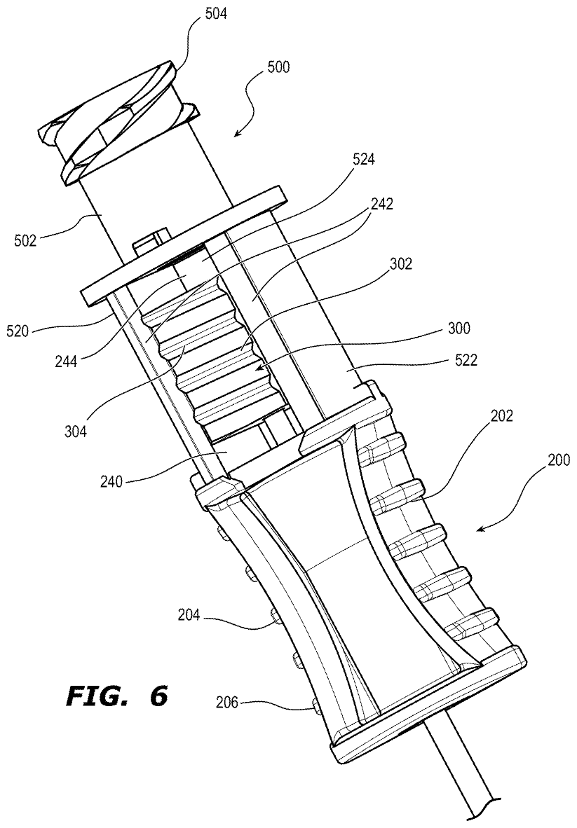

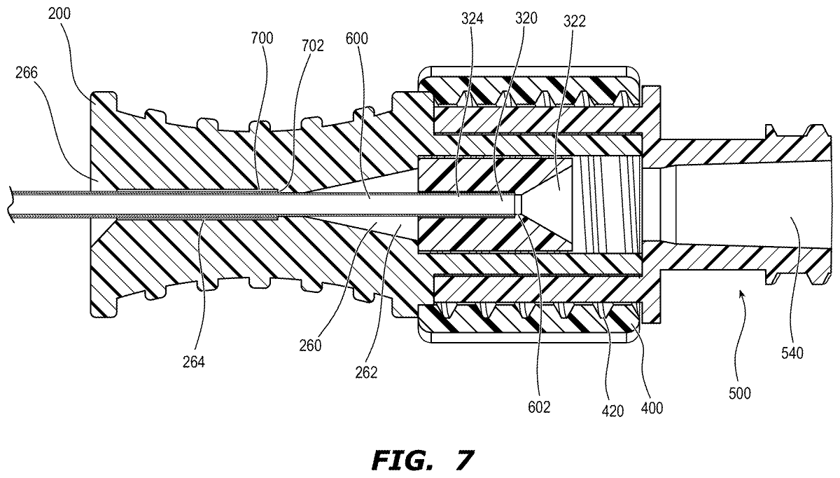

FIGS. 6 and 7 illustrate an exemplary embodiment of first conduit 600, second conduit 700, and handle 200, including body portion 202, slot portion 240, bore portion 260, translating portion 300, and adapter portion 500. In the illustrated embodiment, protrusions 242 of handle 200 and protrusions 522 of adapter portion 500 couple together to merge slot spaces 244 and 524. Thus, in some embodiments, this combined space of 244 and 524 houses translating portion 300 such that translating portion 300 is free to translate axially with respect to the handle of the device, yet may be fixed in the other two dimensions, as well as rotationally.

Adapter portion 500 may comprise a body portion 502 comprising threads 504 that may serve to couple a second medical device. In some embodiments this second medical device may be an elongate medical device that couples to the handle via threads 504. Bore portion 540 may serve as the beginning of a continuous lumen for the second, elongate medical device to pass through the steerable introducer device 100.

FIG. 7 illustrates an exemplary embodiment of first conduit 600, second conduit 700, and handle 200, including body portion 202, slot portion 240, bore portion 260, translating portion 300, actuating portion 400, and adapter portion 500. In the illustrated embodiment, the handle 200 is coupled together such that actuating portion 400 is free to rotate in either direction about the longitudinal axis of the medical device, but portion 400 is translationally fixed in all three dimensions with respect to the handle 200 and device 100. As actuating portion 400 is rotated in either rotational direction around the longitudinal axis of the device by a user of the device, threads 420 of the actuating portion engage threads 304 of translating portion 300, and translate translating portion 300 distally or proximally with respect to the handle body portion 202, along the longitudinal axis of the device. As translating portion 300 of the device is fixedly coupled to first conduit 600, rotating actuating portion 400 has the end effect of displacing first conduit 600 distally or proximally with respect to the other components of the device, including handle 200 and second conduit 700.

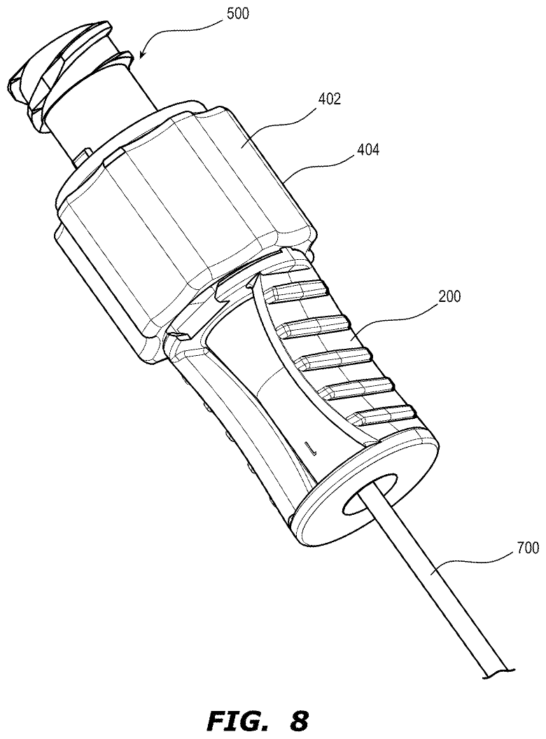

FIG. 8 illustrates an exemplary embodiment of second conduit 700 and handle 200, including body portion 202, actuating portion 400, and adapter portion 500. In the embodiment shown in FIG. 8, the exterior 402 of actuation portion 400 comprises ridges 404 to help facilitate rotation of actuation portion 400 by a user. One of ordinary skill in the art, having the benefit of this disclosure, will be able to devise various mechanisms for a user to provide a rotational actuation force to and/or through actuation portion 400.

FIG. 9 shows an exemplary embodiment of the distal end of the steerable introducer device 100, including the distal end 660 of first conduit 600 and the distal end 780 of second conduit 700. As seen in the embodiment of FIG. 9, the two conduits may be joined using protrusion 662 of the first conduit 600, and cut portion 760 of the second conduit 700. Thus, the distal end of the steerable introducer device 100 may be semi-rigid. The two conduits may be welded together, although one skilled in the art, having the benefit of this disclosure, would be able to devise multiple ways to join the two conduits.

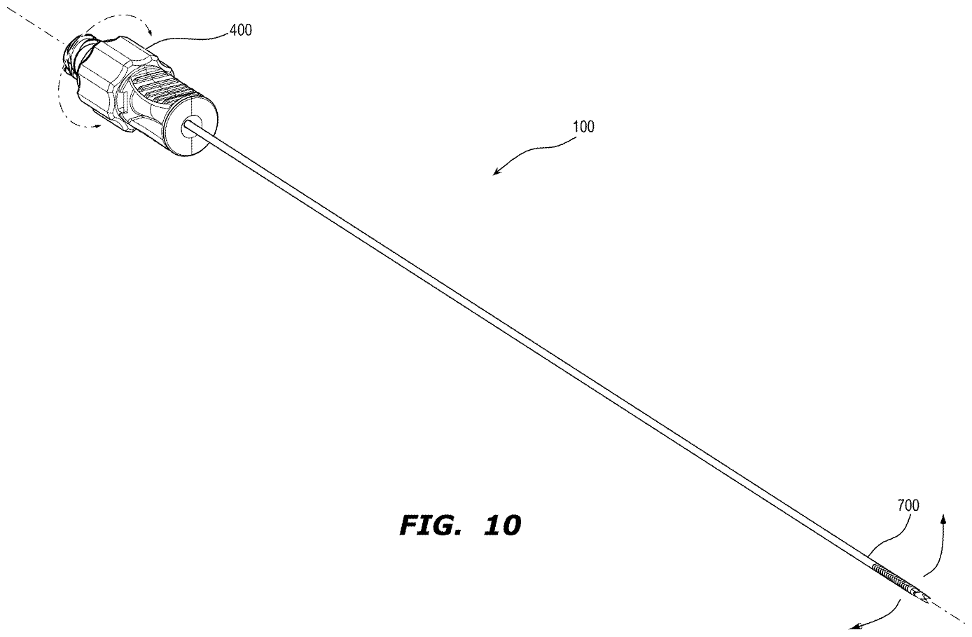

FIG. 10 illustrates an exemplary embodiment of the steerable introducer device 100, including the directions in which the actuation portion 400 may be rotated, and the produced articulation at the distal end of the introducer device 100.

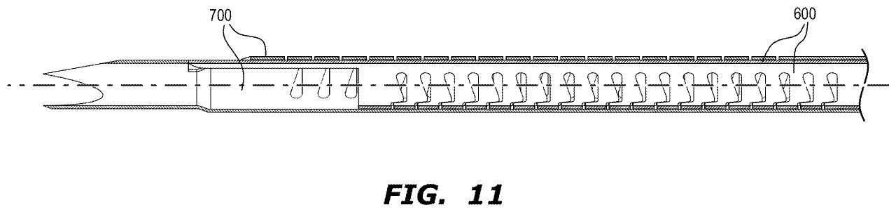

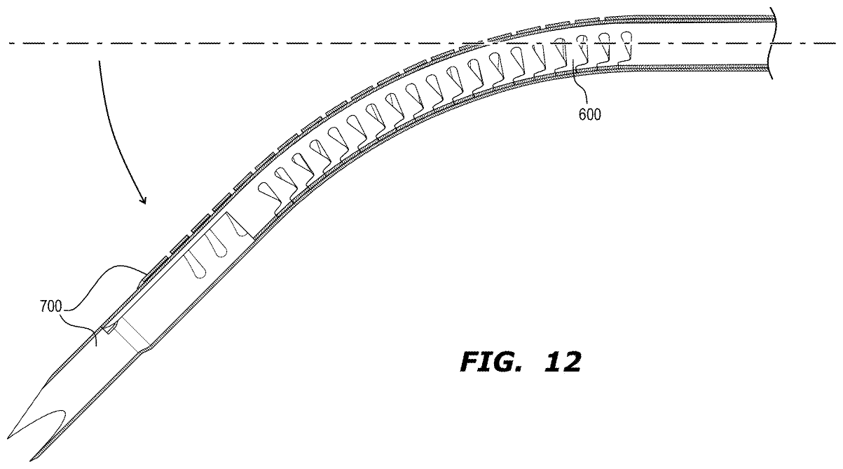

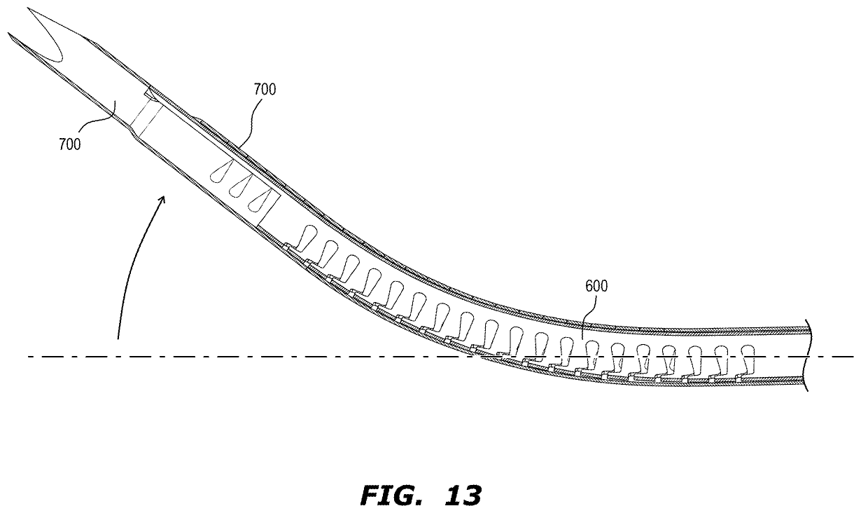

FIGS. 11-13 illustrate an exemplary embodiment of the distal end of the steerable introducer device 100. FIG. 11 illustrates an exemplary embodiment of the introducer device 100 in the linear, or unarticulated configuration. FIG. 12 illustrates an exemplary embodiment of the introducer device 100 articulated in a first direction. FIG. 13 illustrates an exemplary embodiment of the introducer device 100 articulated in a second direction, opposite the first direction.

As further seen in the embodiment shown in FIGS. 11-13, first conduit 600 may be concentrically, and housed entirely, within second conduit 700. Slots 640 of the first conduit 600, and slots 740 of the second conduit 700 may overlap longitudinally, or they may overlap along a length of the introducer device 100. As further seen in the embodiments of FIGS. 11-13, slots 640 of the first conduit 600, and slots 740 of the second conduit 700 may also be disposed radially opposite each other.

As shown in FIG. 12, a compression force may be applied by the user to first conduit 600, through rotation of the actuation portion 400 by the user and distal displacement of the attached translating portion 300. The effect from this action is that this compression force is transmitted as a tensile force on second conduit 700 at the point where the two conduits are joined. This tensile force on second conduit 700 will cause the second conduit 700 to expand in the longitudinal direction. Due to the slots 740 along the side wall of main body portion 720, the main body portion 720 will expand at different rates. Namely, when the tensile force is applied to conduit 700, the side wall with slots 740, and function portion 744 in particular, will expand more than the radially opposite, uncut sidewall, which may expand negligibly or imperceptibly. This interaction between the applied force and slots 740 may cause the second conduit 700 to articulate in a first direction.

In a similar but opposite manner to second conduit 700, the compression force on first conduit 600 may cause the main body 620 of first conduit 600 to contract in the longitudinal direction. Similarly, yet opposite, to the effect explained above, the sidewalls of first conduit 600 may contract at different rates. The sidewall comprising the slots 640, and function portion 644 in particular, may contract more than the uncut sidewall opposite the slots, which may contract negligibly or imperceptibly. This disparity, and interaction between the compression force and slots 640, may cause the first conduit 600 to articulate in the same, first direction as second conduit 700.

As shown in FIG. 13 a tensile force may be applied by the user to first conduit 600, through rotation of the actuation portion 400 by the user and proximal displacement of the attached translating portion. The effect from this action is that the tensile force is transmitted as a compression force on second conduit 700. This compression force on second conduit 700 will cause the second conduit 700 to contract in the longitudinal direction. Due to the slots 740 along the side wall of main body portion 720, the main body portion 720 will contract at different rates. Namely, when the compression force is applied to conduit 700, the side with slots 740, and function portion 744 in particular, will contract significantly more than the radially opposite, uncut side, which may contract negligibly or imperceptibly. This interaction between the applied force and the slots 740 may cause the second conduit 700 to articulate in a second direction.

In a similar but opposite manner to second conduit 700, the tensile force on first conduit 600 may cause the main body 620 of first conduit 600 to expand in the longitudinal direction. Similarly, yet opposite, to the effect explained above, the sidewalls of first conduit 600 may expand at different rates. The sidewall comprising the slots 640, and function portion 644 in particular, may expand significantly more than the uncut sidewall opposite the slots, which may expand negligibly or imperceptibly. This disparity, and interaction between the tensile force and the slots 640, may cause the first conduit 600 to articulate in the same, second direction as second conduit 700.

With reference to the methods and functions explained in FIGS. 11-13, the non-linear configuration, bend, or rate of articulation, may be discretized via the rotation of actuating portion 400. For example, in some embodiments, the further actuating portion 400 is rotated, the greater the degree of articulation at the distal end of the device 100, and vice versa.

Using this method and control over articulation of the introducer device, a user may guide, or steer, the introducer device as it is being inserted into a patient. Thus, through controlling the actuation portion 400, in conjunction with rotating the entire device 100 with respect to a patient, a user may accurately steer the distal end of the device 100 to the desired location for further medical procedures.

In an example of such a procedure, using one embodiment of the device, introducer device 100 may be navigated toward a point of a lesion or target location, and using the articulable distal tip, be navigated and/or maneuvered in such a way as to avoid a bodily structure and/or target a specific portion of a location, or portion of a lesion. Thus, due to its articulating nature, greater control and maneuverability can be exercised with introducer device 100.

In a further embodiment and method of use for the device, introducer device 100 may be navigated toward a lesion or target site, and using the articulable distal end, be repositioned in such a way as to introduce multiple medical instruments, including multiple biopsy devices, at different locations and/or orientations within the same lesion, or target site.

Similarly, in a separate embodiment and method of use, introducer device 100 may be navigated toward multiple lesions, or target sites, and using the articulable distal end, be maneuvered in such a way as to obtain multiple biopsy samples, or introduce multiple medical instruments, at different target sites, such as target sites offset from each other along an arc of articulation of the introducer device 100.

In connection with this, and other, embodiments and methods of obtaining a biopsy and introducing medical instruments, introducer device 100 may function separately from a biopsy device, or other introducible medical instruments. For example, the distal end of the introducer device 100 may be navigated to a first desired location, independent of a biopsy device or introducible medical instrument. After this navigation phase, a biopsy device, or other introducible medical instrument, may be displaced from the proximal end to the distal end of the introducer device 100, through the inner lumen of introducer device 100. Thus, movement and maneuverability of the steerable introducer device 100 may be separate from the second and distinct act of introducing a medical instrument.

With reference to a lesion, after navigating the steerable introducer device to the desired location and distally displacing a biopsy device through the introducer device to retrieve the biopsy sample, the biopsy device may be returned and removed from the inner lumen of introducer device 100, out of the body of the patient. Following this first biopsy retrieval, the distal end of introducer device 100 may be navigated to second desired location, after which the original or a second biopsy device may be inserted into the introducer device to take a second biopsy at the second desired location. This process may be repeated in a similar manner with multiple desired locations.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated by one of skill in the art with the benefit of this disclosure that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the present disclosure.

1. A medical device for introducing medical instruments, comprising:
a first conduit comprising a plurality of slots along a first side of an articulating distal portion;
a second conduit comprising a plurality of slots along a first side of an articulating distal portion;
wherein the first conduit is coupled to the second conduit at a distal end of the first conduit; and
a handle coupled to the first and second conduits, the handle comprising:
an actuating portion, configured to rotate about the longitudinal axis of the device and located distal of the proximal end of the device, the actuating portion configured to displace a distal end of the device from a linear configuration to an articulated configuration when actuated by a user.

2. The medical device of claim 1, wherein the handle further comprises a translating portion configured to couple to the actuating portion.

3. The medical device of claim 2, wherein rotational movement of the actuating portion by a user is configured to translate the translating portion in an axial direction of the device.

4. The medical device of claim 3, wherein the translating portion is coupled to the proximal end of the first conduit.

5. The medical device of claim 1, wherein axial movement of the first conduit is configured to displace a distal ends of the first and second conduit from a linear configuration to an articulated configuration.

6. The medical device of claim 1, wherein the first conduit is within a bore of the second conduit, and is configured to move axially with respect to the second conduit.

7. The medical device of claim 1, wherein the first conduit is coupled to the second conduit adjacent a distal end of the second conduit.

8. The medical device of claim 1, wherein the plurality of slots of the first conduit longitudinally overlap the plurality of slots of the second conduit.

9. The medical device of claim 1, wherein the plurality of slots of the first conduit are radially interior to the plurality of slots of the second conduit.

10. The medical device of claim 1, wherein the plurality of slots of the first conduit, are disposed radially opposite the plurality of slots from the second conduit.

11. The medical device of claim 1, wherein rotation of the actuating portion of the handle by a user in a first rotational direction is configured to articulate the distal end of the device in a first direction.

12. The medical device of claim 11, wherein rotation of the actuating portion of the handle by a user in a second rotational direction is configured to articulate the distal end of the device in a second direction.

13. The medical device of claim 1, wherein the distal end of the second conduit further comprises a cutting tip.

14. The medical device of claim 1, wherein a continuous lumen extends from a proximal end of the device, through the handle and first and second conduits, to a distal end of the device.

15. The medical device of claim 1, wherein a second, elongate medical device is inserted through the proximal end of the first medical device, and is directed through the first medical device, out of the distal end of the first medical device.

16. A method for placing a medical device introducer into a patient's body, comprising:
obtaining a medical device for introducing medical instruments, comprising:
a first conduit comprising a plurality of slots along a first side of an articulating distal portion;
a second conduit comprising a plurality of slots along a first side of an articulating distal portion;
wherein the first conduit is coupled to the second conduit at a distal end of the first conduit; and
a handle coupled to the first and second conduits, the handle comprising:
an actuating portion, configured to rotate about the longitudinal axis of the device and located distal of the proximal end of the device, the actuating portion configured to displace a distal end of the device from a linear configuration to an articulated configuration when actuated by a user;
inserting the medical device for introducing medical instruments, into a patient's body;
rotating the actuating portion of the handle to articulate the distal end of the device such that the device may be directed in a non-linear direction within the patient's body; and
navigating the medical device for introducing medical instruments to a target site within the patient's body.

17. The method of claim 16, wherein rotating the actuating portion of the handle to articulate the distal end of the device, further comprises rotating the actuating portion of the handle in a first direction to articulate the distal end of the device in a first direction.

18. The method of claim 17, wherein rotating the actuating portion of the handle to articulate the distal end of the device further comprises rotating the actuating portion of the handle in a second direction to articulate the distal end of the device in a second direction.

19. The method of claim 16, further comprising repositioning the medical device for introducing medical instruments, to a different location within the same target site, after the target site has already been navigated to.

20. The method of claim 19, further comprising repositioning the medical device for introducing medical instruments, to a location within an additional target site, after the current target site has already been navigated to.
